(19) United States
(12) Patent Application Publication
(10) Pub. No.: US 2011/0152694 A1
(43) Pub. Date: Jun. 23, 2011

(54) BLOOD-PRESSURE SENSOR, MANUFACTURING METHOD THEREOF, AND BLOOD-PRESSURE SENSOR SYSTEM

(75) Inventors: Isao SHIMOYAMA, Tokyo (JP); Kiyoshi MATSUMOTO, Tokyo (JP); Tetsuo KAN, Tokyo (JP); Eiji IWASE, Cambridge, MA (US); Asuto KOYAMA, Tokyo (JP); Yoshiharu AJIKI, Hachioji-shi (JP)

(73) Assignees: OLYMPUS CORPORATION, Tokyo (JP); THE UNIVERSITY OF TOKYO, Tokyo (JP)

(21) Appl. No.: 12/894,814

(22) Filed: Sep. 30, 2010

(30) Foreign Application Priority Data
Dec. 22, 2009 (JP) 2009-291274

(51) Int. Cl. A61B 6/00

(52) U.S. Cl. 600/480

(57) ABSTRACT

A blood-pressure sensor is constituted of an elastic body which is fitted to a blood-vessel outer wall, and a shape of which is deformed by force generated by pulsing motion of expansion and contraction of the blood vessel, and a plurality of nanosized particles dispersedly provided in the elastic body, and when the force is applied to the sensor in a state where the sensor is irradiated with light, the magnitude of the force is measured on the basis of intensity of scattered light from the particles or emission intensity of fluorescence from the particles, the intensity of the scattered light or emission intensity of the fluorescence corresponding to a change in distance between the particles.

### CROSS-REFERENCE TO RELATED APPLICATIONS

This application is based upon and claims the benefit of priority from prior Japanese Patent Application No. 2009-291274, filed Dec. 22, 2009, the entire contents of which are incorporated herein by reference.

### BACKGROUND OF THE INVENTION

1. Field of the Invention

The present invention relates to a blood-pressure sensor to which a dynamic sensor for optically measuring dynamical characteristics of an object such as displacement and force is applied, and which is configured to measure intravascular pressure from the displacement of the blood vessel, manufacturing method thereof, and blood-pressure sensor system.

2. Description of the Related Art

In general, as a blood-pressure monitoring method associated with blood vessel disease such as a congestive heart disease, arteriosclerosis, and the like, and a blood-pressure monitoring method to be employed after replacement of a vessel with an artificial blood vessel, an implantable blood-pressure sensor capable of measuring blood pressure at all times is proposed. In the proposals made so far, the sensor is fixed to the blood-vessel wall, and measurement is invasively carried out, and hence generation of a blood clot or injury to the blood-vessel wall is feared. As a method different from the above, a method of noninvasively inferring the blood pressure by winding a cuff around the blood vessel from outside the vessel, and measuring the internal pressure of the cuff is under consideration. However, when the cuff is actually used, it is not easy to arrange the sensor, and the reliability of the accuracy is not satisfactory. Furthermore, a signal line for transmitting data from the sensor in the body to the outside, and power supply line for supplying electric power to the sensor are necessary. As a result of this, the range of action is greatly restrained, and hence the method is not suitable for ongoing blood-pressure monitoring.

Further, in, for example, each of Documents 1 and 2, a blood-pressure monitoring method using micro-electromechanical system (MEMS) sensors such as a pressure sensor, strain sensor, and the like requiring a sensor internal power source for carrying out power supply is proposed. Document 1 is Micheal A. Fonseca, Mark G. Allen, Jason Kroh and Jason White, Flexible wireless passive pressure sensors for biomedical applications, Solid-State Sensors, Actuators, and Microsystems Workshop, pp. 37-42 (2006). Document 2 is P. Cong, Darrin J. Young, and Wen H. Ko, Wireless Less-Invasive Blood Pressure Sensing Microsystem for Small Laboratory Animal in vivo Real-Time Monitoring, 2008 5th International Conference on Networked Sensing Systems (2008).

A blood-pressure sensor which requires no wiring connection for power supply to the blood-pressure sensor or extraction of detection signal of an unmovably installed power source or control apparatus, can be carried, carries out blood-pressure measurement of an objective blood vessel on a noncontact basis, and requires no power supply, a manufacturing method thereof, and a blood-pressure sensor system are demanded.

### BRIEF SUMMARY OF THE INVENTION

According to an embodiment of the present invention, there is provided a blood-pressure sensor characterized by comprising: a sensor main body fitted to a blood-vessel outer wall, and formed of an elastic body a shape of which is deformed by force generated by pulsing motion of expansion and contraction of the blood vessel; and a plurality of nanosized particles dispersedly provided in the sensor main body, wherein when the force is applied to the sensor main body in a state where the sensor main body is irradiated with light, the magnitude of the force is measured according to intensity of response light from the particles, the intensity of the response light corresponding to a change in distance between the particles.

Further, the present invention provides a manufacturing method of a blood-pressure sensor comprising: coating a flat substrate with a resist, and forming a plurality of trenches extending in an arbitrary direction in a form of juxtaposed lines, and having a width of a nanosized particle; applying a particle-dispersion liquid in which a plurality of particles are contained, and continuously feeding the particles into the trenches by using the Template Assisted Self-Assembly (TASA) method to densely arrange the particles in line in such a manner that each of the particles is partially exposed; coating the surface of the substrate on which the particles are arranged with a liquidized elastic material in a vacuum atmosphere, and then curing the elastic material; and peeling off the resist away from the substrate, and separating a sensor section formed of a cured elastic body to which the particles densely arranged in the trenches are adhered, and the substrate from each other.

Additionally, the present invention provides a blood-pressure sensor system comprising: a sensor section constituted of an elastic body which is fitted to a blood-vessel outer wall, and a shape of which is deformed by force generated by pulsing motion of expansion and contraction of the blood vessel, and a plurality of nanosized particles dispersedly provided in the elastic body; a light source configured to irradiate the sensor section with predetermined light; and a measuring device configured to receive response light of the light, the response light returning from the sensor section, and corresponding to a change in distance between the particles, and measure the magnitude of force applied to the elastic body according to a change in intensity of the response light.

Advantage of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a view showing the external configuration of a sensor section used for a blood-pressure sensor of an embodiment according to the present invention.

FIG. 2 is a graph showing changes in intensity of scattered light when both ends of the sensor section are pulled.

FIGS. 3A, 3B, and 3C are views each showing a conceptual configuration example of the sensor section using nanosized particles of fluorescent beads.

FIG. 4 is a view showing a conceptual configuration example of a blood-pressure sensor system using the sensor section.

FIG. 5A is a view showing an example of the external configuration of a portable blood-pressure sensor system to which a fitting attachment for fitting the above-mentioned blood-pressure sensor system to a radius is attached, and FIG. 5B is a block diagram showing a configuration example of a portable blood-pressure sensor system.

FIG. 6 is a view for explaining a function of the blood-pressure sensor system.

FIG. 7 is a view showing the state where the arrangement is configured in order that the reflected light may not be measured to detect only the scattered light.

FIG. 8 is a graph showing the relationship between the pressure detected by the sensor section and amount of a change in emission intensity.

FIGS. 9A, 9B, 9C, 9D, and 9E are views for explaining the formation process of the sensor section.

### DETAILED DESCRIPTION OF THE INVENTION

Hereinafter, an embodiment of the present invention will be described below in detail with reference to the drawings.

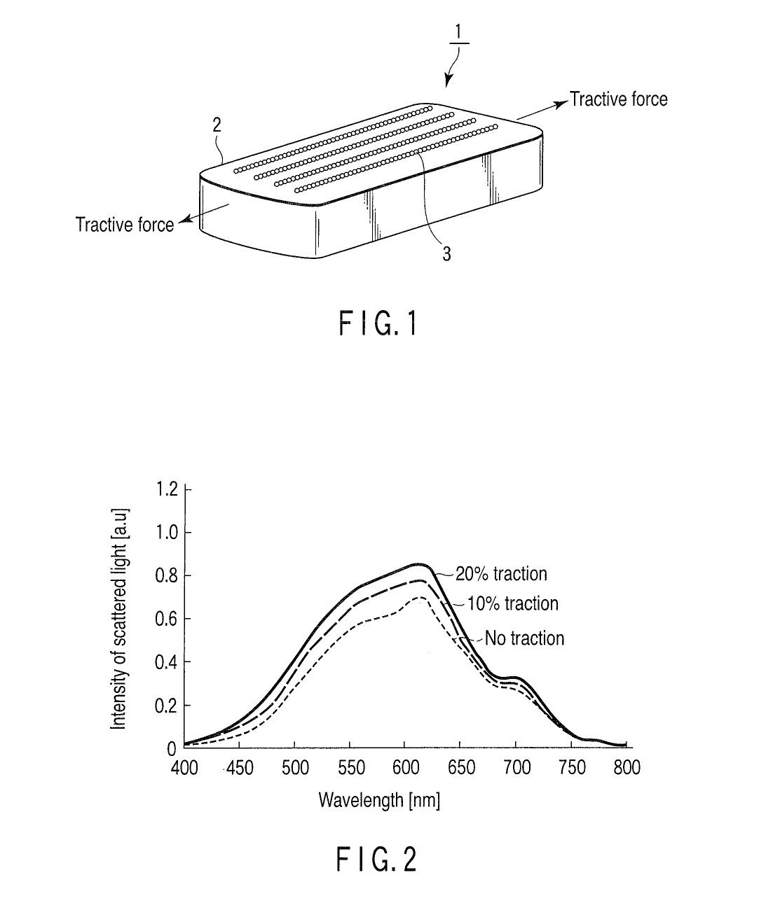

In FIG. 1, the external configuration of a sensor section used for a blood-pressure sensor of an embodiment according to the present invention is shown.

The sensor section 1 is constituted of an elastic body 2 previously having a predetermined elastic coefficient, and a plurality of particles (particle body) 3 fixed to the surface of the elastic body 2 in line in a pattern to be described later or dispersedly contained in the elastic body 2. In the sensor section 1, distances between particles are changed by tractive force or pressure (pressing force) applied at both ends or at one end thereof.

The elastic body 2 has, in this embodiment, a shape of a rectangular parallelepiped, and as an elastic body material, a silicon series elastic body (silicon elastomer) such as polydimethylsiloxane (PDMS) or the like is used. It should be noted that when as the particle 3 to be contained in the elastic body 2, a quantum dot or fluorescent nanoparticle to be described later is used, and the light to be applied is visible light, the elastic body 2 is formed of an elastic member through which desired light is transmitted in order that the applied light and emitted light can be transmitted through the elastic body 2. For example, when QDOT® (registered trade name: made by Invitrogen Corp.) is taken as an example, a combination of QDOT® 605 (excitation wavelength λ Aex=400 [nm], fluorescence wavelength λ Aem=605 [nm]) and QDOT® 705 (excitation wavelength λ Bex=605 [nm], fluorescence wavelength λ Bem=705 [nm]) is desirable.

The particle 3 has, for example, a spherical shape, and is a nanosized particle. As the particle 3, a nanoparticle of a metal such as gold, silver, aluminum, and the like or fluorescent nanoparticle formed by causing silica (silicon dioxide) to adsorb a fluorescent dye or further semiconductor nanoparticle such as a silicon quantum dot, and the like can be used. In this embodiment, although a description will be given by taking a spherical particle 3 as an example, when particles of a certain shape are arranged on the surface of the elastic body 2 in a predetermined pattern or when the particles are dispersed inside the elastic body 2, if the particles can emit light like in the case of the spherical shape, the shape of the particles may be that of a rectangular parallelepiped, polyhedron, petrosa, cylindrical body, barrel-shaped body or the like. Further, when distribution differences in the amount of light responding to the design are required, the distribution differences can be realized by adjusting the dispersion amount of the particles 3.

A function of the blood-pressure sensor according to this embodiment will be described below.

In the blood-pressure sensor, when force is applied to the elastic body 2, intervals between the arranged particles 3 change, whereby changes in the optical/electromagnetic resonance states between the particles are caused. The changes are measured by using a method such as spectroscopy or the like.

In the sensor section 1, the obtained light (response light) differs depending on the arranged particles (metallic nanoparticles, fluorescent nanoparticles or semiconductor nanoparticles). Hereinafter, particles of the different types will be described below.

(1) An example in which a metal is used for the nanoparticles, for example, Au nanoparticles will be described below. In a state where the sensor section 1 is irradiated with white light, when the distances d between the particles are widened to a certain degree in directions in which the particles are separated from each other due to tractive force applied to the sensor section 1, intensity of the scattered light of the response light increases (and the maximum absorption wavelength is shifted toward the shorter wavelength side by the surface plasmon effect). By monitoring the intensity of the scattered light, it is possible to sense the displacement d1. By the force-displacement relationship in the elastic body 2 in which the displacement d1 and elastic coefficient are known values, it is possible to measure the force applied to the sensor section 1.

FIG. 2 is a view showing results obtained by measuring changes in the intensity of the scattered light when both the ends of the sensor section 1 is inserted in the traction section of a stretcher (not shown) to be fixed, and tractive force [no traction (0% traction), 10% traction, and 20% traction] is applied thereto. From the above results, it can be seen that when tractive force is applied to the sensor section 1 at least from one side thereof, and the stronger the tractive force, the higher the intensity of the scattered light is increased. It should be noted that 10% traction implies a state where the length of the elastic body 2 is prolonged by 10% of the length thereof by the traction (state where the length thereof has become 1.1) assuming that the length L of the transparent elastic body 2 shown in FIG. 1 in the longitudinal direction thereof is 1.

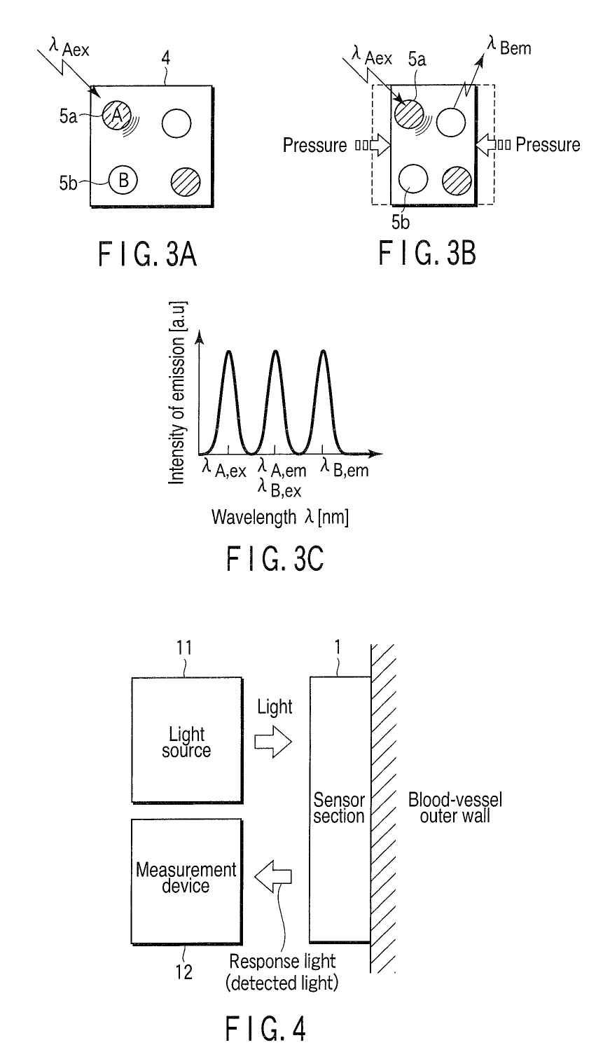

(2) The case where fluorescent nanoparticles or semiconductor nanoparticles are used as the nanoparticles will be described below with reference to FIGS. 3A to 3C. Here, as the fluorescent nanoparticle, a fluorescent bead can be employed, and as the semiconductor nanoparticle, a quantum dot can be employed.

The structure of the sensor section 1 is a structure in which fluorescent beads 5 (having a size identical with the particles 3) are dispersed in an elastic body 4 (equivalent to the elastic body 2) such as PDMS or the like. The fluorescent beads 5 include particles 5a and 5b having different excitation/fluorescence wavelengths. It should be noted that in FIGS. 3A and 3B, although two types of fluorescent beads are used as the nanosized particles, the nanosized particles are not limited to these, and a plurality of types of fluorescent beads having excitation/fluorescence wavelengths different from each other may be included in the fluorescent beads 5.

In FIG. 3C, the particle 5a has a bell-shaped light-emitting characteristic in which the excitation wavelength spectrum (response light) centers at λ_{Aex}, and fluorescent spectrum centers at λ_{Aem}. On the other hand, λ_{Aem} to λ_{Bex} are selected in order that the excitation wavelength spectrum of the particle 5b may overlap the fluorescent spectrum of the particle 5a.

FIG. 3A shows the state where no pressure is applied, the particle 5a and particle 5b are not in close vicinity to each other, even when the particle 5a emits fluorescent light, the fluorescent light is hardly transmitted to the particle 5b, and the particle 5b emits no fluorescent light. However, as shown in FIG. 3B, when pressure is applied at least from one side, the distance between the particles 5a and 5b becomes smaller, and hence the fluorescent light of the particle 5a is transmitted to the particle 5b, whereby the particle 5b emits fluorescent light. Accordingly, by continuously monitoring the emission intensity of the wavelength of λ_{Bem}, it is possible to measure, from outside, the pressure applied to the elastic body 4.

As described above, when the sensor section 1 is used for pressure measurement, it is not necessary to supply energy for detection into the sensor section 1. Accordingly, the shape of the sensor section 1 has to be made into a sheet-like form, and the sheet-like sensor section 1 has only to be wound around the blood vessel which is the object to be tested, and it is not necessary to connect detection wiring or batteries to the sensor section itself. By previously irradiating the sensor section 1 with predetermined light, and detecting a change (change in light intensity) in the response light, it is possible to utilize the sensor section 1 as a pressure sensor (blood-pressure sensor).

Furthermore, by imparting anisotropy to the rigidity of the deformation, and using fluorescent beads sets of different wavelengths for the sensors of different axes directions, it is easily possible to realize polyaxial detection directions of the sensor section. To the sensor section in which the two functions are different from each other, predetermined light is applied, and when the response light corresponds to the aforementioned item (1), the light is scattered light, and when the response light corresponds to the item (2), it is light emission. It is possible to recognize whether the applied force is tractive force or pressure from the response light.

Accordingly, when the sensor section 1 is, for example, wound around the blood-vessel outer wall to be fitted thereto, by utilizing the measuring of the blood pressure from the degree of generated distortion, it is possible to realize a contactless blood-pressure sensor requiring no power supply to the sensor itself.

Next, FIG. 4 shows a conceptual configuration example of a blood-pressure sensor system using the aforementioned sensor section 1.

This system is constituted of the sensor section 1, a light source 11, and measuring device 12 configured to observe the emission intensity of the response light from the sensor section 1.

When the configuration is that using fluorescent beads for the sensor section 1, as the light source 11, a light source including an excitation wavelength spectrum λ_{Aex} is suitable. Further, the measuring device 12 measures a wavelength of λ_{Bem} which is the fluorescent wavelength spectrum of the particle 5b. On the other hand, when the configuration is that using metallic nanoparticles for the sensor section 1, as the light source 11, white light of a light source such as a halogen lamp is suitable. Further, it is desirable that the measuring device 12 be a device capable of measuring a wavelength the scattering intensity of which is the maximum, for example, a wavelength of about 600 [nm] in the example of FIG. 2. The combination of these can be suitably selected.

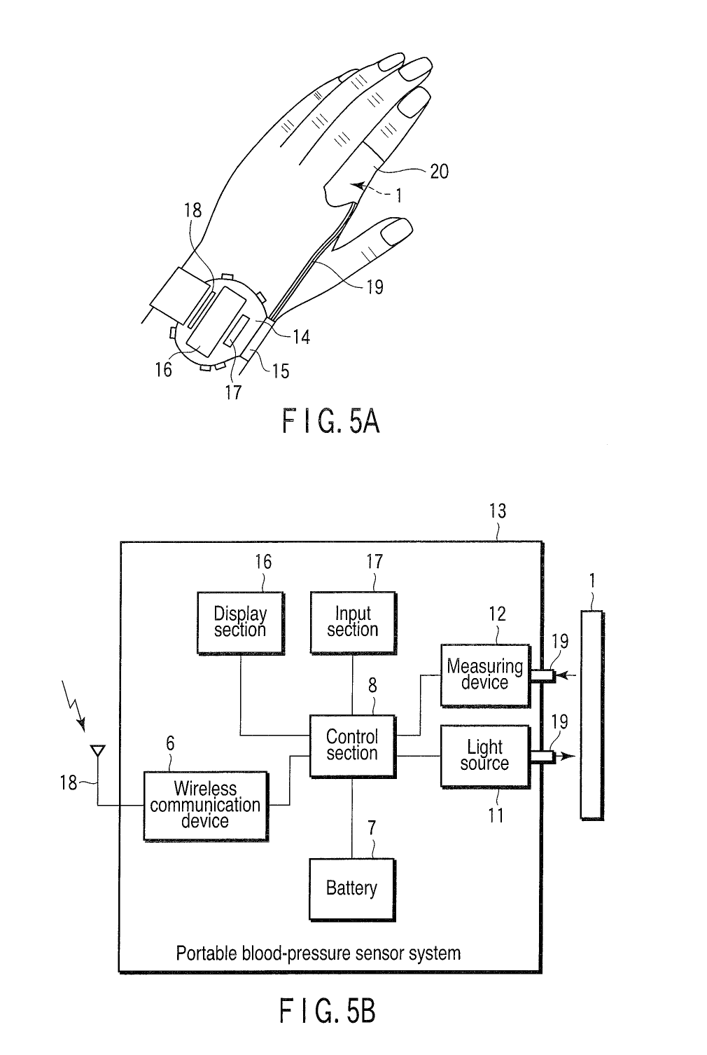

Next, FIG. 5A shows an example of the external configuration of a portable blood-pressure sensor system to which a fitting attachment for fitting the above-mentioned blood-pressure sensor system to a radius is attached, and FIG. 5B is a block diagram showing a configuration example of a portable blood-pressure sensor system.

This portable blood-pressure sensor system 13 is an example of that of a wristwatch type to be used in the case where blood pressure is measured by means of the sensor section 1 implanted in such a manner that the sensor section 1 is in contact with, for example, an artery (blood-vessel outer wall) of a radius of the hand. The portable blood-pressure sensor system 13 is constituted of a case (main body section) 14 integrally containing therein the aforementioned light source 11, a measuring device 12, wireless communication device 6, battery 7, and control section 8, and fitting attachment configured to fit and fix the case 14 to the arm, such as a belt 15. On the surface of the case 14, a display section 16 configured to display measurement results of the blood pressure and the like, operation instruction, and setting contents is provided. In the vicinity of the display section 16, an input section 17 configured to carry out input of setting or instructions, and antenna section 18 for communication are arranged. The display section 16, input section 17, and communication processing, and the like are controlled by the control section 8. Further, optical fibers 19 configured to irradiate the implanted sensor section 1 with light of the light source 11, and receive the response light at the measuring device 12 is provided to extend from the case 14. These optical fibers 19 are fixed to the finger in order that the optical fibers 19 may not deviate from the sensor section 1 by a fastening device 20 such as a supporter, belt or the like. In this example, although a small button battery or the like is assumed as the battery to be contained in the main body section 14, alternatively, a solar cell may also be used. Further, the system may be a system configured to establish a power source by means of the radio wave by utilizing the wireless communication device.

According to this portable blood-pressure sensor system, if only the system is within the communication area of the wireless communication device, free action is enabled, restriction on the sphere of action is greatly improved, and the system is also suitable for continuous blood-pressure monitoring to be carried out for a long period.

Here, although the blood-pressure sensor system attached to the radius has been described as an example, the blood-pressure system is not limited to this, and it is also easy to attach the system to a blood vessel of other part such as an upper arm, leg or the like in the same manner. Further, if size reduction of the main body section 14 in which the light source 11, measuring device 12, wireless communication device, and battery are incorporated can be realized, it is also conceivable that the system can be configured to a size of a finger ring.

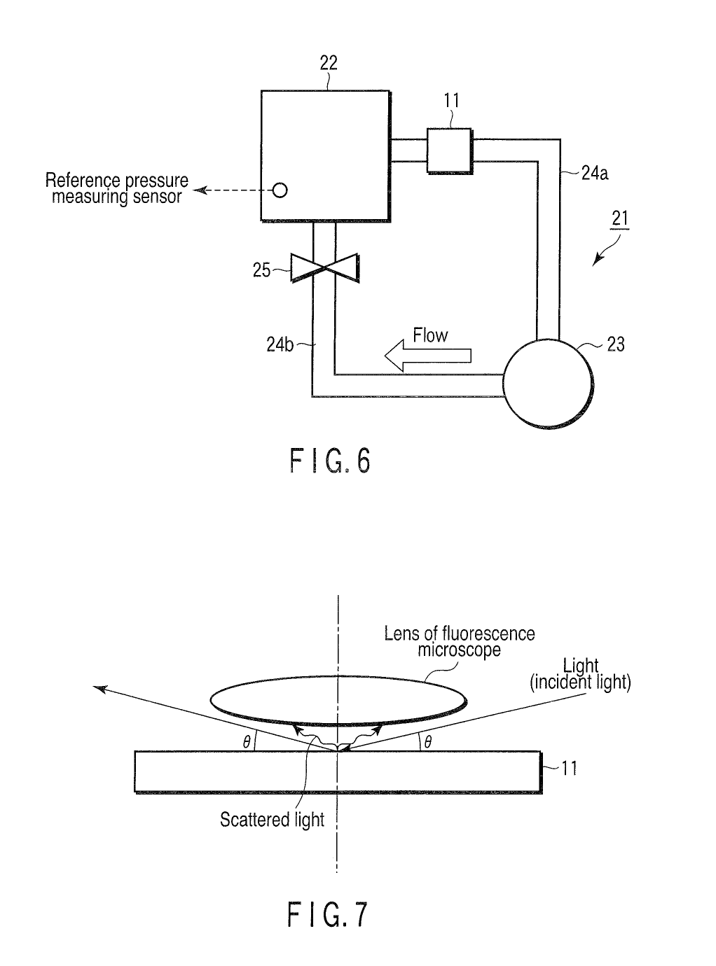

Next, a function of the blood-pressure sensor system of this embodiment will be described below with reference to FIG. 6.

Here, an example in which the blood-pressure sensor is fitted to a blood vessel (main artery circulatory model 21) or the like which is the object to be tested, and acquisition of data of pressure in the simulated blood vessel is carried out from deformation (expansion and contraction) of the blood-vessel outer wall will be described below. The main artery circulatory model 21 is constituted of a compliance tank 22, circulating pump 23, simulated blood vessels 24a and 24b serving as flow paths connecting these members to each other, sensor section 1 provided to be wound around the outer wall of the simulated blood vessel 24a, and flow-path resistance 25 provided in the middle of the simulated blood vessel 24b. In this circulatory path, simulated blood is circulated in a pulsing manner by the circulating pump. A reference pressure measuring sensor (not shown) is provided in the compliance tank 22 for the purpose of pressure value estimation.

In the sensor system, measurement was carried out by using, for example, a white light source (halogen lamp, JCR 12V-100W manufactured by USHIO) as the light source 11, and using a fluorescence microscope (Olympus, IX51) as the measuring device 12. As the particles to be contained in the sensor section 1, Au nanoparticles (absorption wavelength 600 nm) were used.

As shown in FIG. 7, here, in order to detect only the scattered light, the arrangement is configured in such a manner that the reflected light is not measured. That is, arrangement is made in such a manner that light is caused to obliquely enter from the outside of a lens of the fluorescence microscope 26, and the reflected light derived from the incident light does not enter the lens.

Concomitantly with the incidence of the light, the scattered light generated by the particles is not dependent on the incidence angle, and hence is made incident on the lens to be condensed. By monitoring the intensity of the scattered light resulting from the condensed reflected light by using the fluorescence microscope 26, the relationship between the displacement of the sensor section 1 and the change in emission intensity was obtained. Further, on the basis of the displacement and elastic coefficient of the elastic body 2, the applied force can be estimated. Thus, the force applied to the sensor section 1 is derived from the emission intensity. As the reference, simulated blood pressure data in the simulated blood vessel was also acquired by using the reference pressure measuring sensor.

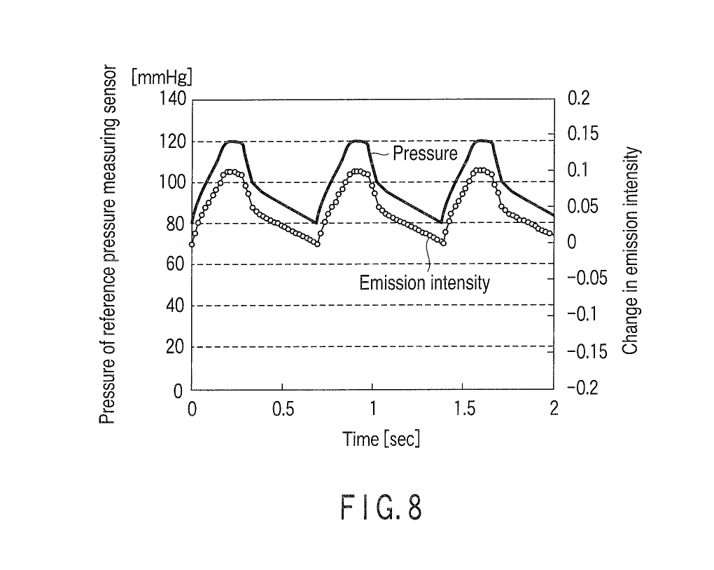

In FIG. 8, as results of the above, the relationship between the pressure detected by the sensor and the change in emission intensity with time is shown. From the result, there is a correlation between the change in emission intensity and the blood pressure data, and it is possible to estimate the blood pressure data from the change in emission intensity by using the correlation coefficient.

Thus, according to the blood-pressure sensor system of this embodiment, information on the pulsation (blood pressure) in the blood vessel can be obtained by analyzing light responding to the scattered light from the blood-pressure sensor, and hence it is possible to carry out blood-pressure measurement in a noncontact manner without supplying driving power to the sensor. Accordingly, unlike in the conventional case, wiring connection between the fixedly installed power source and sensor section is not required, and it is possible to easily construct a portable system shown in FIG. 5.

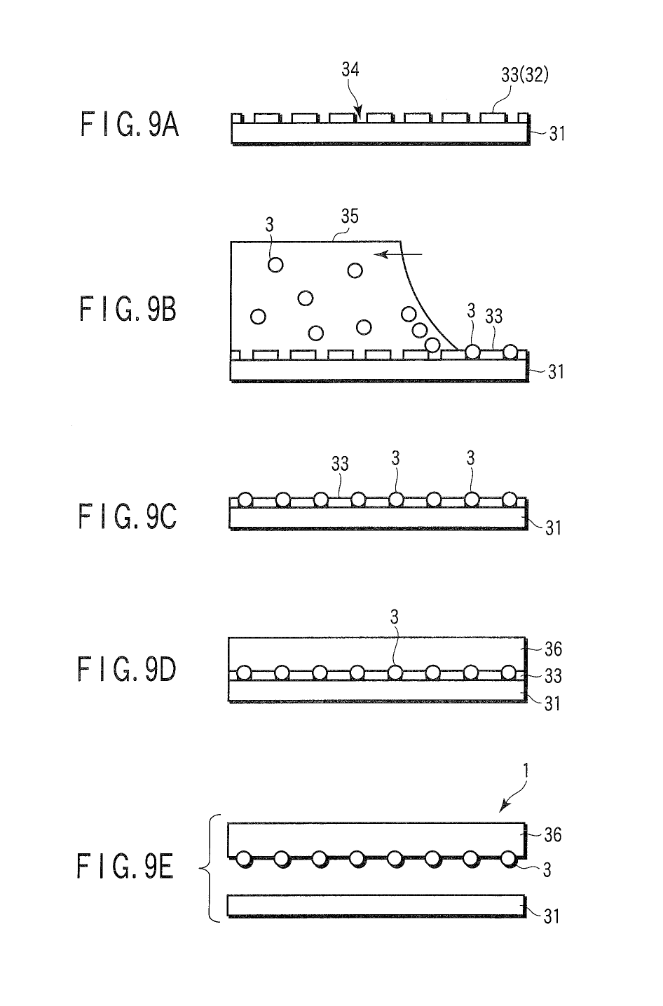

The formation process of the sensor section 1 will be described below with reference to FIGS. 9A to 9E.

A description will be given by taking the structure in which a plurality of particles 3 are dispersed in or arranged on the elastic body 2 shown in FIG. 1 as an example. It should be noted that although the sensor section 1 can also be formed by mixing nanosized particles into a PDMS gel, in this embodiment, in order to form the sensor section 1 into a desired pattern, the following manufacturing method is proposed.

First, as shown in FIG. 9A, a resist 32 is coated to a hard and flat substrate 31 such as a silicon substrate, glass substrate, ceramic substrate or the like, and a resist pattern 33 is formed by a direct writing method such as electron beam lithography using an electron beam (EB). It is desirable that the resist pattern 33 to be drawn should have a specific shape in an arbitrary direction, such as a linear line-and-space shape shown in FIG. 1, for example, a pattern extending in one direction in a form of a plurality of lines. In this embodiment, a plurality of trenches 34 are assumed. As the width of the trenches, a width of a size equal to the particle diameter in which the nanosized particle 3 to be arranged can be contained without play is suitable. It is desirable that the space between the trenches 34 be about 100 to 300 nm. Although all the spaces are basically identical with each other, the spaces may be appropriately changed according to the design/specification. Further, this sensor has high sensitivity to force parallel to the direction of the lines, and hence the structure formed by taking the fact into consideration is desirable. For example, when the deformation of the blood vessel is to be measured, it is desirable that the sensor be arranged perpendicular to the longitudinal direction (blood flow direction) of the blood vessel.

In this embodiment, a depth of the trench 34 is made slightly smaller than the diameter of the particle 3, and the particle 3 is fitted into the trench 34 in such a manner that a top part of the particle 3 is exposed to the outside of the trench 34. That is, the configuration is set in such a manner that when force is applied to the elastic body 2 from outside, the particle 3 is hardly caused to dart out of the trench 34 by being embedded in the resin material with the center of the particle 3 slightly deeper than the surface of the resin material at the time of completion of the elastic body 2. It should be noted that FIG. 9A shows a cross-sectional structure in the direction in which the plurality of trenches are traversed.

Further, in this embodiment, although the resist pattern 33 is formed by the direct writing method, the method is not limited to this, and it is possible to form an identical pattern (in this case, the line-and-space structure) by using a patterning method based on exposure using a mask, and development.

Subsequently, as shown in FIG. 9B, a particle-dispersion liquid 35 in which a large number of particles 3 are contained is applied. At this time, the particles 3 are continuously fed into the trenches 34 to be densely arranged in line by using the Template Assisted Self-Assembly (TASA) method or the like using a template having the line-and-space structure. More specifically, the particle-dispersion liquid 35 is dropped onto the line-and-space structure. Although a meniscus crosses the trenches 34 concomitantly with the evaporation of the dispersion liquid 35, at this time, particles 3 collected at the distal end part of the meniscus are trapped into the trenches 34, whereby it is possible to obtain self-arranged particle arrays as shown in FIG. 9C.

Then, a silicon elastomer 36 such as PDMS or the like which is an elastic material is sufficiently mixed with a hardening agent, and thereafter the resultant is subjected to vacuum defoaming in such a manner that there are no remaining air bubbles inside. As shown in FIG. 9D, the silicon elastomer 36 is flatly applied to the surface of the substrate on which the nanoparticles are arranged in a vacuum, and is then cured. When the silicon elastomer 36 is applied, the silicon elastomer 36 adheres to the exposed top part of each particle 3, and the particles 3 are fixed to the silicon elastomer 36 side, i.e., the elastic body 2 side concomitantly with the curing. It should be noted that when the application is carried out in the air, there is the possibility of the silicon elastomer 36 being prevented from entering the nanopattern by the obstruction of air remaining at the junction plane. Thus, by applying the silicon elastomer 36 in the vacuum atmosphere, the air remaining at the junction plane prevents the silicon elastomer 36 from entering the silicon nanopattern.

Subsequently, as shown in FIG. 9E, the above resultant is submerged in the resist removing liquid to remove the resist pattern 33, thereby peeling off the silicon elastomer 36 (elastic body 2) adhering to the nanoparticles 3 away from the substrate 31. By the manufacturing process described above, the sensor section 1 can be manufactured.

As described above, according to the manufacturing method of this embodiment, by utilizing a template using a resist, and having the line-and-space structure, it is possible to easily disperse or arrange nanoparticles in or on the PDMS.

The appearance shape of the elastic body functioning as the sensor section main body can be formed by application, and hence the elastic body has a high degree of freedom with respect to the thickness and shape, and can be easily formed into a desired shape.

As described above, according to the embodiment of the present invention, it is possible to provide a blood-pressure sensor which requires no wiring connection for power supply to the blood-pressure sensor or extraction of detection signal of an installed power source or control apparatus, can be carried, carries out blood-pressure measurement of an objective blood vessel on a noncontact basis, and requires no power supply, manufacturing method thereof, and blood-pressure sensor system.

The present invention includes the following aspects:

(1) A sensor characterized by comprising:

a sensor main body formed of an elastic body a shape of which is deformed by application of external force; and

a plurality of nanosized particles which are densely dispersed in the sensor main body in a pattern extending in an arbitrary direction in a form of lines arranged at intervals in such a manner that the particles are partially exposed, wherein

when the external force is applied to the sensor main body in a state where the sensor main body is irradiated with light, the magnitude of the external force is measured according to intensity of scattered light from the particles, the intensity of the scattered light corresponding to a change in distance between the particles.

(2) A sensor characterized by comprising:

a sensor main body formed of an elastic body a shape of which is deformed by application of external force; and

a plurality of types of nanosized particles which are contained in the sensor main body in a dispersed and mixed state, and possess different excitation/fluorescence wavelengths, wherein

when the external force is applied to the sensor main body in a state where the sensor main body is irradiated with light, the magnitude of the external force is measured according to emission intensity of fluorescence emitted from the particles according to a change in distance between the particles.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A blood-pressure sensor comprising:
a sensor main body fitted to a blood-vessel outer wall, and formed of an elastic body a shape of which is deformed by force generated by pulsing motion of expansion and contraction of the blood vessel; and
a plurality of nanosized particles dispersedly provided in the sensor main body, wherein
when the force is applied to the sensor main body in a state where the sensor main body is irradiated with light, the magnitude of the force is measured according to intensity of response light from the particles, the intensity of the response light corresponding to a change in distance between the particles.

2. The sensor according to claim 1, wherein
the particles are densely dispersed in the sensor main body in a pattern extending in an arbitrary direction in a form of lines arranged at intervals in such a manner that a top part of each of the particles is exposed, and when tractive force resulting from the expansion is applied to the sensor main body in a state where the sensor main body is irradiated with light, the magnitude of the force is measured according to intensity of scattered light from the particles, the intensity of the scattered light corresponding to a change in distance between the particles.

3. The sensor according to claim 2, wherein
the particle is a nanosized particle formed of a metallic material.

4. The sensor according to claim 1, wherein
the particles are comprised of a plurality of types of nanosized particles possessing different excitation/fluorescence wavelengths, and are contained in the sensor main body in a dispersed and mixed state, and
when pressure resulting from the contraction is applied to the sensor main body in a state where the sensor main body is irradiated with light, the magnitude of the force is measured according to emission intensity of fluorescence emitted from the particles, the emission intensity of the fluorescence corresponding to a change in distance between the particles.

5. The sensor according to claim 4, wherein
the particle is a nanosized particle formed of a fluorescent material or a semiconductor material.

6. A manufacturing method of a blood-pressure sensor comprising:
coating a flat substrate with a resist, and forming a plurality of trenches extending in an arbitrary direction in a form of juxtaposed lines, and having a width of a nanosized particle;
applying a particle-dispersion liquid in which a plurality of particles are contained, and continuously feeding the particles into the trenches by using the Template Assisted Self-Assembly (TASA) method to densely arrange the particles in line in such a manner that each of the particles is partially exposed;
coating the surface of the substrate on which the particles are arranged with a liquidized elastic material in a vacuum atmosphere, and then curing the elastic material; and
peeling off the resist away from the substrate, and separating a sensor section formed of a cured elastic body to which the particles densely arranged in the trenches are adhered, and the substrate from each other.

7. A blood-pressure sensor system comprising:
a sensor section constituted of an elastic body which is fitted to a blood-vessel outer wall, and a shape of which is deformed by force generated by pulsing motion of expansion and contraction of the blood vessel, and a plurality of nanosized particles dispersedly provided in the elastic body;
a light source configured to irradiate the sensor section with predetermined light; and
a measuring device configured to receive response light of the light, the response light returning from the sensor section, and corresponding to a change in distance between the particles, and measure the magnitude of force applied to the elastic body according to a change in intensity of the response light.

8. The sensor system according to claim 7, further comprising:
a portable case configured to contain therein the light source, and the measuring device; and
optical fibers extending from the case, and configured to optically connect the light source and the measuring device to the sensor section.
